(19) United States
(12) Patent Application Publication
(10) Pub. No.: US 2019/0255292 A1
(43) Pub. Date: Aug. 22, 2019

(54) DEFLECTABLE SHEATH WITH INFLATABLE BALLOON

(71) Applicant: Oscor Inc., Palm Harbor, FL (US)

(72) Inventors: Thomas P. Osypka, Palm Harbor, FL (US); Jörg Teßarek, Lingen (DE); Andrew Enerson, Hudson, FL (US); Chet Michael, Tampa, FL (US)

(21) Appl. No.: 16/278,625

(22) Filed: Feb. 18, 2019

Related U.S. Application Data
(60) Provisional application No. 62/710,436, filed on Feb. 16, 2018.

(51) Int. Cl. A61M 25/01; A61M 25/10

(52) U.S. Cl. CPC A61M 25/0147; A61M 25/0136; A61M 2025/09008; A61M 25/10; A61M 2025/015; A61M 25/0113

(57) ABSTRACT

A steerable intravascular catheter includes a handle assembly having opposed proximal and distal end portions and defining a longitudinal axis therebetween. An elongated sheath extends from the distal end portion of the handle assembly, has opposed proximal and distal end portions, and includes a tubular body wall forming a central lumen for accommodating the introduction of a device and a fluid lumen radially outward from and parallel to the central lumen. The distal end portion of the elongated sheath is deflectable relative to the proximal end portion of the elongated sheath. A rotatable actuation assembly is associated with the handle assembly for controlling deflection of the distal end portion of the elongated sheath. An inflatable occlusion balloon is positioned on an outer surface of the distal end portion of the elongated sheath. The fluid lumen of the elongated sheath is in fluid communication with an interior of the balloon.

### CROSS-REFERENCE TO RELATED APPLICATIONS

This application claims the benefit of and priority to U.S. Provisional Patent Application No. 62/710,436 filed Feb. 16, 2018, which is incorporated by reference herein in its entirety.

### BACKGROUND OF THE INVENTION

### 1. Field of the Invention

The subject invention relates to intravascular catheters, and more particularly, to a guided intravascular catheter device having an inflatable balloon mounted on its distal end and a steering assembly for accurately placing the distal end of the sheath and balloon at a targeted location in a patient's body.

### 2. Description of Related Art

There are instances where physicians must introduce diagnostic and therapeutic devices into the body, such as diagnostic and therapeutic electrodes, ultrasound transducers and other surgical tools. The diagnostic and therapeutic devices are often carried by catheters, which allow physicians to gain access to the body in a minimally invasive manner by way of bodily lumens. In cardiac treatment, for example, a catheter is advanced through a main vein or artery into the region of the heart that is to be treated.

One method of introducing diagnostic and therapeutic devices into the body is to introduce a tubular member (typically a “catheter sheath”) into the vicinity of the targeted region. A diagnostic or therapeutic catheter device is then passed through the sheath to the targeted region. If necessary, the diagnostic or therapeutic catheter device may be removed after its function is performed, but the sheath can be left in place, so that other catheters or other devices can be advanced to the targeted region to complete the diagnostic and/or therapeutic procedure. One such device commonly advanced to the targeted region through the catheter sheath is a balloon occlusion catheter. Balloon occlusion catheters can be used to occlude vessels to temporary block up a vessel to then deploy contract media and or a drug to a certain location inside the human body or vascular system. Traditional balloon occlusion catheters can be introduced into the vascular system through a central lumen of the catheter sheath.

Catheter sheaths can be steerable. Examples of steerable sheaths and devices are disclosed in commonly assigned U.S. Pat. Nos. 9,498,602 and 9,572,957 to Osypka et al., and U.S. Patent Application Publication No. 2015/0057610 to Osypka et al. While these devices are well suited for the precise placement of diagnostic or therapeutic devices within a patient's body, these steerable sheath devices do not include a balloon for treatment.

There is a need, therefore, for an improved guiding sheath with a distally mounted inflatable balloon, which allows the distal section of the sheath to be deflected, is easy to navigate as a delectable guiding sheath, is efficient to fabricate and easy to use.

### SUMMARY OF THE INVENTION

A steerable intravascular catheter includes a handle assembly having opposed proximal and distal end portions and defining a longitudinal axis therebetween. An elongated sheath extends from the distal end portion of the handle assembly and has opposed proximal and distal end portions. The elongated sheath includes a tubular body wall forming a central lumen for accommodating the introduction of a device and a fluid lumen radially outward from and parallel to the central lumen. The distal end portion of the elongated sheath is deflectable relative to the proximal end portion of the elongated sheath. A rotatable actuation assembly is operatively associated with the handle assembly for controlling deflection of the distal end portion of the elongated sheath. An inflatable occlusion balloon is positioned on an outer surface of the distal end portion of the elongated sheath. The fluid lumen of the elongated sheath is in fluid communication with an interior of the balloon.

In accordance with some embodiments, the steerable intravascular catheter includes an inflation port positioned on the handle assembly in fluid communication with the fluid lumen allowing the inflatable occlusion balloon to be inflated and deflated.

The elongated sheath can include a pull-wire lumen radially outward from and parallel to the central lumen. The steerable intravascular catheter can include an elongated pull-wire extending through the pull-wire lumen of the elongated sheath and terminating within the distal end portion of the elongated sheath. It is contemplated that the elongated pull-wire can have a proximal end operatively connected to the handle assembly and a distal end anchored to the distal end portion of the elongated sheath. In some embodiments, the steerable intravascular catheter includes a pull-wire anchor ring mechanically coupling a distal end of the elongated pull-wire to the distal end portion of the elongated sheath.

The distal end portion of the elongated sheath can be made from a softer material than the proximal end portion of the elongated sheath to accommodate deflection. The elongated sheath can define a circumference and a predetermined usable length (UL) extending from the proximal end portion of the elongated sheath substantially to the distal end portion of the elongated sheath. The predetermined UL can range from 30 cm to 120 cm.

The rotatable actuation assembly can include a rotatable control knob operatively connected to a proximal end of the elongated pull-wire. Rotation of the rotatable control knob can pull or release the elongated pull-wire and can cause the distal end portion of the elongated sheath to deflect away from the longitudinal axis or back toward the longitudinal axis. The handle assembly can include a drive mechanism for actuating the elongated pull-wire in response to bi-directional angular rotation of the rotatable control knob. The bi-directional angular rotation of the rotatable control knob about the longitudinal axis of the handle assembly can effectuate reciprocal axial movement of the elongated pull-wire and corresponding angular deflection of the distal end portion of the elongated sheath.

In accordance with some embodiments, the handle assembly can include a hemostatic valve operatively connected to the central lumen designed to minimize blood loss and prevent air embolisms. The handle assembly can include a luer type locking connection on a proximal end of the central lumen. The handle assembly can include a flush port in fluid communication with the central lumen to flush the central lumen. The proximal end portion of the elongated sheath can extend entirely through the handle assembly and terminates at a sealed access port communicating with the central lumen defined by the tubular body wall.

These and other features of the systems and methods of the subject disclosure will become more readily apparent to those skilled in the art from the following detailed description of the embodiments taken in conjunction with the drawings.

### BRIEF DESCRIPTION OF THE DRAWINGS

So that those skilled in the art to which the steerable intravascular catheter of the subject invention appertains will readily understand how to make and use the device without undue experimentation, preferred embodiments thereof will be described in detail herein below with reference to certain figures, wherein:

FIG. 1A is a schematic perspective view of a steerable intravascular catheter constructed in accordance with an embodiment of the subject invention, showing an inflatable occlusion balloon mounted on the distal end portion of an elongated sheath;

FIG. 1B is a schematic perspective view of the proximal end of the steerable intravascular catheter of FIG. 1A, showing the hemostatic valve; and

FIG. 2 is a schematic cross-sectional view of the steerable intravascular catheter of FIG. 1A, showing the pull-wire lumen and fluid lumen.

### DETAILED DESCRIPTION OF THE PREFERRED EMBODIMENTS

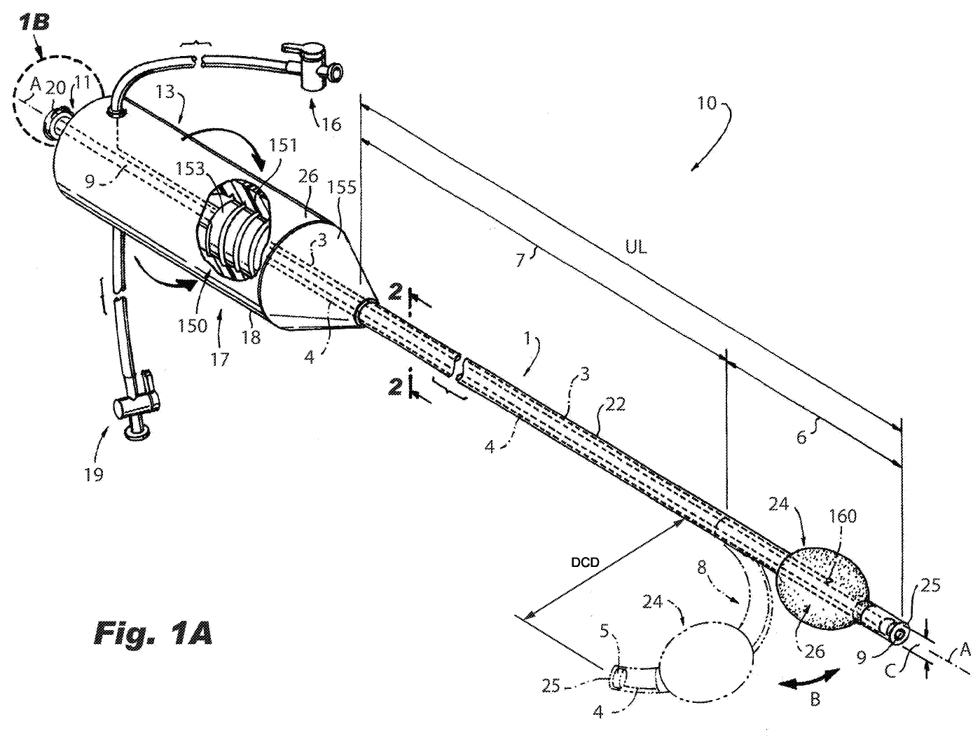

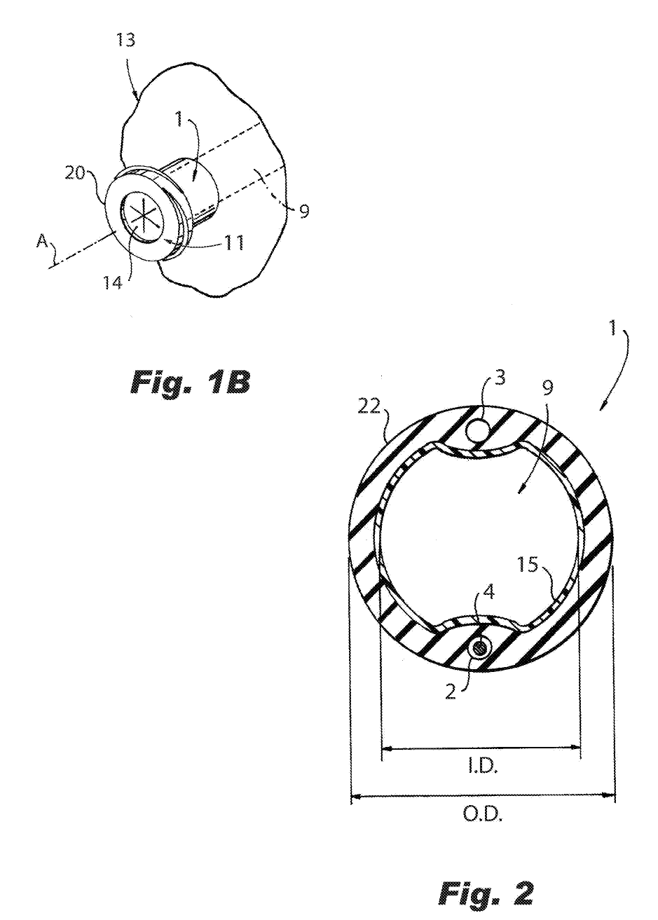

Reference will now be made to the appended drawings wherein like reference numerals identify similar structures or features of the subject invention. For purposes of explanation and illustration, and not limitation, there is illustrated in FIG. 1A a new and useful steerable intravascular catheter constructed in accordance with a preferred embodiment of the subject invention and designated generally by reference numeral 10. Other embodiments of steerable intravascular catheter 10 in accordance with the disclosure, or aspects thereof, are provided in FIGS. 1B-2, as will be described. Steerable intravascular catheter 10 is adapted and configured to facilitate the intracardiac, renal and/or peripheral placement of diagnostic and therapeutic devices during a surgical procedure.

As shown in FIG. 1A, a steerable intravascular catheter 10 includes a handle assembly 13 having opposed proximal and distal ends defining a longitudinal axis A therebetween. An elongated sheath 1 extends from the distal end portion of handle assembly 13. Elongated sheath 1 has opposed proximal and distal end portions and includes a tubular body wall 22. Distal end portion 6 of elongated sheath 1 is deflectable relative to proximal end portion 7 of elongated sheath 1. Deflectable distal end portion 6 of elongated sheath 1 is made from a softer material than the proximal end portion (e.g. the stiffer sheath section 7) of elongated sheath 1 to accommodate deflection. Handle assembly 13 includes a rotatable actuation assembly 17 for controlling deflection of deflectable distal end portion 6 of elongated sheath 1. An inflatable occlusion balloon 24 is positioned on an outer surface of deflectable distal end portion 6 of elongated sheath 1. Elongated sheath 1 defines a circumference C and a predetermined usable length (UL) extending from the start of proximal end portion 7 of elongated sheath 1 by handle assembly 13 substantially to the distal most end of distal end portion 6 of elongated sheath 1. The predetermined UL can range from 30 cm to 120 cm.

Procedures such as the endovascular treatment of peripheral occlusions with mechanical aspiration/thrombectomy systems are made more efficient and easier to perform with steerable sheath device 10. The combination of elongated sheath 1, mounted inflatable occlusion balloon 24, and the ability to mechanically deflect distal tip portion 6 to appropriately steer the system into the correct target vessel allow for an increase in efficiency over traditional catheter sheaths.

As shown in FIGS. 1A and 2, tubular body wall 22 defines a central lumen 9 and a fluid lumen 3 radially outward from and parallel to central lumen 9. Fluid lumen 3 of elongated sheath 1 is in fluid communication with an interior 26 of inflatable occlusion balloon 24. Fluid lumen 3 is schematically shown as a dashed line in FIG. 1A for the sake of clarity. Those skilled in the art will readily appreciate that fluid lumen 3 is tubular shaped and extends within tubular body wall 22 from a longitudinal position proximate to an inflation port 16, along the length of elongated sheath 1, to a port 160 defined in tubular body wall 22 within interior 26 of balloon 24. Inflation port 16 is positioned on handle assembly 13 in fluid communication with fluid lumen 3 allowing inflatable occlusion balloon 24 to be inflated and deflated. Those skilled in the art will readily appreciate that a connecting tube or the like can extend from fluid lumen 3 in tubular body wall 22 to inflation port 16 To inflate balloon 24, an inflation fluid, such as saline solution or a contrast medium, is supplied to interior 26 of balloon 24 through inflation port 16 using an inflation syringe, or the like. To deflate balloon 24 the inflation syringe can provide a pulling vacuum to interior 26 of balloon 24 through inflation port 16 and balloon 24 returns to its deflated state.

With continued reference to FIGS. 1A and 2, elongated sheath 1 includes a pull-wire lumen 2 radially outward from and parallel to central lumen 9. Steerable sheath device 10 includes an elongated pull-wire 4 extending through pull-wire lumen 2 of elongated sheath 1 and terminating within distal end portion 6 of elongated sheath 1. For sake of clarity, FIG. 1A only shows elongated pull-wire 4, without pull-wire lumen 2. Those skilled in the art will readily appreciate that, in the embodiment shown in the figures, pull-wire lumen 2 has a tubular shape and extends within tubular body wall 22 from a longitudinal position proximate a distal end of a manually rotatable control knob 18, described in more detail below, and down along the length of elongated sheath 1 to a pull-wire anchor ring 5. Elongated pull-wire 4 is positioned within pull-wire lumen 2 and has a proximal end that extends out of pull-wire lumen 2 and is operatively connected to handle assembly 13 and a distal end anchored to distal end portion 6 of elongated sheath 1 at pull-wire anchor ring 5. Pull-wire anchor ring 5 mechanically couples a distal end of elongated pull-wire 4 to distal end portion 6 of elongated sheath 1. In the embodiment of FIG. 1A, pull-wire anchor ring 5 is mounted proximate to a distal tip 25 of distal end portion 6.

With continued reference to FIG. 1A, manually rotatable control knob 18 of rotatable actuation assembly 17 is operatively connected by way of a drive mechanism 150 to a proximal end of elongated pull-wire 4. The manual rotation of rotatable control knob 18 pulls or releases elongated pull-wire 4 by way of drive mechanism 150, described below, and causes distal end portion 6 of elongated sheath 1 to deflect away from longitudinal axis A or back toward longitudinal axis A. Handle assembly 13 includes drive mechanism 150 for actuating elongated pull-wire 4 in response to bi-directional angular rotation of rotatable control knob 18, as described in more detail below.

As shown in FIG. 1A, drive mechanism includes a worm gear 153 mounted for reciprocal longitudinal movement within the interior cavity of handle assembly 13 relative to elongated sheath 1. Drive mechanism 150 further includes an axially rotatable drive nut 151 meshed with threads of worm gear 153 for effectuating the reciprocal longitudinal movement of worm gear 153. Rotatable control knob 18 is directly connected to drive nut 151 in the interior cavity of handle assembly 13. Rotatable control knob 18 can be configured for gripping and rotation by a user to rotate drive nut 150 and move worm gear (e.g. work coil) 153. When drive nut 150 is rotated by way of rotation of rotatable control knob 18, worm gear 153 rotates and moves longitudinally in either the distal or proximal direction. A distal end portion 155 of handle assembly 13 is fixed relative to elongated sheath 1, such that rotatable control knob 18 can be rotated with respect thereto.

With continued reference to FIG. 1A, in handle assembly 13, pull-wire 4 extends out of tubular body wall 22 near a distal end 26 of manually rotatable control knob 18 so that it can be coupled to worm gear 153. Pull-wire 4 is coupled to worm gear 153, e.g. coupled by way of a set screw, such that the axial translation of worm gear 153 pulls or releases pull-wire 4 thereby causing the deflection of distal end portion 6. In FIG. 1A, worm gear 153 is advanced to a distal position such that worm gear 153 abuts the inner surface of handle assembly 13 such that worm gear 153 cannot be advanced further in the distal direction. This position can be associated with a straight condition of sheath 1 (shown in solid lines). Worm gear 153 can be advanced proximally by rotation of drive nut 151 to pull pull-wire 4 and deflect distal end portion 6 of sheath 1 (as shown in the broken lines). Softer distal sheath end 6 in its deflected position is designated by numeral 8.

Bi-directional angular rotation of rotatable control knob 18 about longitudinal axis A of handle assembly 13 effectuates reciprocal axial movement of elongated pull-wire 4 and corresponding angular deflection of distal end portion 6 of elongated sheath 1, as shown schematically by arcuate arrow B in FIG. 1A. The deflection of the distal end portion 6 can be defined by the deflection curve diameter (DCD), which can range from 7 mm to 50 mm. In some embodiments, distal tip 25 of the distal end portion 6 can be deflected up to 180 degrees, or more. In other words, it can go from facing a distal direction to facing a proximal direction. While shown and described in conjunction with drive mechanism 150, other suitable drive mechansims can be used, e.g. those shown and described in commonly assigned U.S. Pat. Nos. 9,498,602 and 9,572,957 to Osypka et al., and U.S. Patent Application Publication No. 2015/0057610 to Osypka et al., which are all hereby incorporated by reference in their entirety.

As shown in FIGS. 1A-2, the proximal end portion of elongated sheath 1 extends entirely through handle assembly 13 and terminates at a sealed access port 11 communicating with central lumen 9 defined by tubular body wall 22. Handle assembly 13 includes a hemostatic valve 14 operatively connected to central lumen 9 designed to minimize blood loss and prevent air embolisms. Handle assembly 13 includes a luer type locking connection 20, e.g. fitting, on a proximal end of central lumen 9. Handle assembly 13 includes a flush port 19 in fluid communication with central lumen 9 to flush central lumen 9. Central lumen 9 can include a PTFE liner 15. Tubular body 22 of sheath 1 can have an outer diameter (OD) ranging from 6 to 30 French (F). An inner diameter (ID) of tubular body 22 that defines, in-part, central lumen 9 can range from 5 to 26 F. Hemostatic valve 14, luer type locking mechanism 20 and flush port 19 can be similar to those described in commonly assigned U.S. Pat. Nos. 9,498,602, 9,572,957, and 8,974,420 to Osypka et al., and U.S. Patent Application Publication No. 2015/0057610 to Osypka et al., all of which are hereby incorporated by reference in their entirety.

While the steerable intravascular catheter device of the subject invention has been shown and described with reference to preferred embodiments, those skilled in the art will readily appreciate that various changes and/or modifications may be made thereto without departing from the spirit and scope of the subject disclosure.

What is claimed is:

1. A steerable intravascular catheter, comprising:
a) a handle assembly having opposed proximal and distal end portions and defining a longitudinal axis therebetween;
b) an elongated sheath extending from the distal end portion of the handle assembly and having opposed proximal and distal end portions, the elongated sheath including a tubular body wall forming a central lumen for accommodating the introduction of a device and a fluid lumen radially outward from and parallel to the central lumen, wherein the distal end portion of the elongated sheath is deflectable relative to the proximal end portion of the elongated sheath;
c) a rotatable actuation assembly operatively associated with the handle assembly for controlling deflection of the distal end portion of the elongated sheath; and
c) an inflatable occlusion balloon positioned on an outer surface of the distal end portion of the elongated sheath, wherein the fluid lumen of the elongated sheath is in fluid communication with an interior of the balloon.

2. A steerable intravascular catheter as recited in claim 1, further comprising an inflation port positioned on the handle assembly in fluid communication with the fluid lumen allowing the inflatable occlusion balloon to be inflated and deflated.

3. A steerable intravascular catheter as recited in claim 1, wherein the elongated sheath includes a pull-wire lumen radially outward from and parallel to the central lumen.

4. A steerable intravascular catheter as recited in claim 3, further comprising an elongated pull-wire extending through the pull-wire lumen of the elongated sheath and terminating within the distal end portion of the elongated sheath.

5. A steerable intravascular catheter as recited in claim 4, wherein the elongated pull-wire has a proximal end operatively connected to the handle assembly and a distal end anchored to the distal end portion of the elongated sheath.

6. A steerable intravascular catheter as recited in claim 4, further comprising a pull-wire anchor ring mechanically coupling a distal end of the elongated pull-wire to the distal end portion of the elongated sheath.

7. A steerable intravascular catheter as recited in claim 4, wherein the rotatable actuation assembly includes a rotatable control knob operatively connected to a proximal end of the elongated pull-wire, wherein rotation of the rotatable control knob pulls or releases the elongated pull-wire and causes the distal end portion of the elongated sheath to deflect away from the longitudinal axis or back toward the longitudinal axis.

8. A steerable intravascular catheter as recited in claim 7, wherein the handle assembly includes a drive mechanism for actuating the elongated pull-wire in response to bi-directional angular rotation of the rotatable control knob.

9. A steerable intravascular catheter as recited in claim 7, wherein bi-directional angular rotation of the rotatable control knob about the longitudinal axis of the handle assembly effectuates reciprocal axial movement of the elongated pull-wire and corresponding angular deflection of the distal end portion of the elongated sheath.

10. A steerable intravascular catheter as recited in claim 1, wherein the distal end portion of the elongated sheath is made from a softer material than the proximal end portion of the elongated sheath to accommodate deflection.

11. A steerable intravascular catheter as recited in claim 1, wherein the elongated sheath defines a circumference and a predetermined usable length (UL) extending from the proximal end portion of the elongated sheath substantially to the distal end portion of the elongated sheath, wherein the predetermined UL ranges from 30 cm to 120 cm.

12. A steerable intravascular catheter as recited in claim 1, wherein the handle assembly includes a hemostatic valve operatively connected to the central lumen designed to minimize blood loss and prevent air embolisms.

13. A steerable intravascular catheter as recited in claim 1, wherein the handle assembly includes a luer type locking connection on a proximal end of the central lumen.

14. A steerable intravascular catheter as recited in claim 1, wherein the handle assembly includes a flush port in fluid communication with the central lumen to flush the central lumen.

15. A steerable intravascular catheter as recited in claim 1, wherein the proximal end portion of the elongated sheath extends entirely through the handle assembly and terminates at a sealed access port communicating with the central lumen defined by the tubular body wall.
